(19) United States
(12) Patent Application Publication
(10) Pub. No.: US 2013/0030242 A1
(43) Pub. Date: Jan. 31, 2013

(54) Dog anxiety relief bone conduction audio device, system

(71) Applicant: Michael Rolf Ruehring, Manassas, VA (US)

(72) Inventor: Michael Rolf Ruehring, Manassas, VA (US)

(73) Assignee: Michael R. Ruehring, Manassas, VA (US)

(21) Appl. No.: 13/437,985

(22) Filed: Apr. 3, 2012

Related U.S. Application Data
(60) Provisional application No. 61/511,812, filed on Jul. 26, 2011.

(51) Int. Cl. A61M 21/02; A61D 99/00

(52) U.S. Cl. 600/28

(57) ABSTRACT

A waterproof audio device and system that transmits sound via transcutaneous bone conduction provides therapeutic auditory stimulation music to a dog. The device is integrated into an adjustable dog collar component and a removable adjustable muzzle component. Both the dog collar and the muzzle components will be adjustable or available in various sizes to fit any sized dog. The present invention uses commercially available transducers to produce sounds in the low, mid and high frequency ranges. A commercially available sound source for the therapeutic auditory stimulation music signal can also be provided as part of the waterproof audio device. Commercially available fasteners enable the dog's owner to position the device on the neck and head of the dog for providing acceptable sound conduction and individualized fit for the dog.

### CROSS-REFERENCE TO RELATED APPLICATIONS

This patent application claims priority under 35 U.S.C. 119 (e) of the co-pending U.S. Provisional Patent Application Ser. No. 61/511,812, filed Jul. 26, 2011, and titled “Dog anxiety relief bone conduction audio device, system.”

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

1. Field of the Invention

The present invention pertains to the field of animal husbandry. More particularly, the present invention pertains to the field of animal controlling or handling.

2. Background Description

Thunderstorm phobia is a relatively common phobia for dogs, particularly for those that live in areas where thunderstorms are prolific. Shull-Selcer and Stagg (1991) reported that 93% of cases of dogs with noise phobias involved fear of thunder and other loud noises including fireworks. Whether fear of thunderstorms is elicited by a singular traumatic experience or prolonged exposure, the result is often highly distressing for dogs and owners.

In areas where thunderstorms are a common occurrence, there are some dogs that are so badly affected that they have an inability to function for hours before, during, and after a storm. Many thunderstorm phobic dogs adopt self-management strategies that vary from attempting to escape the home, to digging into carpets, seeking out dark den like spaces to hide in, or crawling behind a bathroom sink or toilet. Others will pace back and forth during the storm, unable to focus on owners trying to calm them down. Stress is also manifested through excessive panting, pupil dilation, sweating paws, raised heartbeat, loss of appetite, whimpering, trembling and an inability to settle.

In humans, music therapy has been determined in studies to provide a beneficial effect for patients experiencing extreme stress. The discipline of medical music therapy began after World War II when community musicians went to Veterans Hospitals around the country to play for the thousands of veterans suffering both physical and emotional trauma. The patients' notable physical and emotional responses to music led the doctors and nurses to request that hospitals hire musicians.

During the early 1950s, studies showed beneficial effects of music in surgical patients, such as increased cardiac output and decreased heart rate (HR), respiratory rate, and blood pressure (BP).

Researchers in the 1970s began evaluating the behavioral responses of preterm infants to simple music containing human heartbeat sounds. Findings from studies conducted during this time indicated that premature infants in the neonatal intensive care unit (NICU) benefit from this therapeutic auditory stimulation. Later studies reported that postnatal therapeutic auditory stimulation promoted a cardiac response. When the infant was crying, the HR decreased in response to therapeutic auditory stimulation; during the quiet state, the infant's HR increased in response to therapeutic auditory stimulation. Therapeutic auditory stimulation and music therapy are now widely used in hospitals and other health care facilities throughout the world.

Like human babies, dogs seem to be attracted to the mathematical simplicity of the music, the sound of singing voices, and the familiar human heartbeat sound that is provided by therapeutic auditory stimulation. Studies indicate they learn and are calmed by the same basic relaxation principles of simplicity, repetition, predictability, ordered structure, and consistent tempo that humans are.

In 2003, members of The American Boarding Kennels Association (ABKA) verified that the same therapeutic auditory stimulation used in neo-natal units was effective with animals. Playing auditory stimulation with heartbeat sounds for anxious dogs reduced separation anxiety, diarrhea, aggression and excessive barking. That same year, researchers the Honolulu Zoo claimed they could calm aggressive male chimps to sleep by providing the same auditory stimulation.

Delivery of the above described therapeutic auditory stimulation can be transmitted using any audio system. Audio systems have historically been air conduction systems that rely on tympanic hearing for sound transmission.

In tympanic hearing, the sound travels through the ear canal to the eardrum, making it vibrate. These vibrations are passed to three small bones in the middle ear, the ossicles, by a process called air conduction. These in turn pass the vibrations to the cochlea and the fluid it contains. Movement in this fluid bends the tiny hair cells along the length of the cochlea, generating signals in the auditory nerve. The nerve signals pass to the brain, which interprets them as sound.

Alternatively, bone conduction hearing is when sound vibrations are transmitted directly from the skull and jaw bones to the cochlea, missing the outer and middle ears. While bone conduction devices were originally developed for the hearing impaired and as hearing aid devices, these devices have now been adapted for high fidelity musical signals. Audio systems are predominantly of the air conduction type. In order effectively deliver therapeutic auditory stimulation, air conduction audio systems rely on ear plugs that are placed in the ear or use speakers that transmit sound through the air. For human patients, these methods of sound transmission are acceptable. However, dogs provide exceptional challenges for the air conduction systems.

Dogs will generally not tolerate objects such as an air conductive ear plug being inserted into their ears. Also, unless confined to a small space or a kennel, a dog will likely not remain close enough to an air conduction audio system using external speakers to receive benefit from therapeutic auditory stimulation in the event of a thunderstorm or other noise event. Confining a dog during such an event to keep it close to the audio system could actually lead to increased stress and possible injury to the dog. Additionally, air conduction methods of therapeutic auditory stimulation may not be practical in a public venue, away from the home, outdoors, or in a vehicle. Humans sharing a space with a dog could potentially find the therapeutic auditory stimulation annoying and choose to turn it off. With bone conduction sound transmission, these disadvantages are overcome.

### SUMMARY OF THE INVENTION

It is the object of the present invention to provide an audio device that allows high fidelity therapeutic auditory stimulation music to be delivered to a dog through transcutaneous bone conduction.

A further object of the invention is to provide an integrated audio system that includes both the headphone unit and a signal source unit.

In addition, the present invention is integrated into an adjustable dog collar component and a removable adjustable muzzle component. Both the dog collar and the muzzle components will be adjustable or available in various sizes to fit any sized dog. Dog collar is understood to mean any device designed to be worn around the neck of a dog such as, but not limited to, a collar.

Additionally, an object of the present invention is to enable the dog's owner to position the device on the neck and head of the dog for providing acceptable sound conduction and individualized fit for the dog.

According to the present invention, the foregoing and other objects are achieved in part by having a transducer in contact with the skull of the dog for transmitting musical signals via bone conduction.

The present invention has at least one transducer that is able to transmit transcutaneous sound via bone conduction through the head of the dog. The present invention is functional with at least one transducer, however, at least one transducer should also be understood to include a plurality of transducers. A pre-amp and an amplifier can also be worn on the dog collar of the dog or can be part of a signal source unit to which the transducer or transducers are connected. The pre-amp, amplifier, and the signal source can also be integrated as a part of a transducer. The present invention is intended to be worn on the neck and head of the dog.

The present invention has a further embodiment that integrates the sound source with the sound transmission. This sound source can be in the form of a disk player (e.g., CDs, DVDs, minidiscs, etc.), MP3 player, AM/FM radio, audio transceiver or other such devices known as personal audio devices. The sound source can communicate with the transducers by wireless or wired connection.

Additionally, the present invention will provide a sound source that comes pre-loaded with therapeutic auditory stimulation music incorporating the sound of a human heartbeat.

Finally, the present invention provides a method for positioning the transducers at various locations on the head of the dog. The transducers may be fixed to the dog collar component. In addition, the transducers may be able to slide to different locations around the dog collar component. Finally, the transducers may be able to be removed from the dog collar component and then to be replaced in another location around the dog collar component. As a minimum, the dog's owner should be able to locate the transducer below the dog's ear or on top of the dog's head. By moving the transducers, the dog's owner may improve sound transmission and the dog's comfort for wearing the device.

### BRIEF DESCRIPTION OF THE DRAWINGS

FIG. 1 shows a side view of a dog wearing the dog anxiety relief bone conduction audio device, system with the removable, optional muzzle component attached.

FIG. 2 shows a profile view of a dog anxiety relief bone conduction audio device, system with the removable, optional muzzle component attached.

FIG. 3 shows a side view of a dog wearing the dog anxiety relief bone conduction audio device, system with the removable, optional muzzle component removed.

FIG. 4 shows a profile view of a dog anxiety relief bone conduction audio device, system with the removable, optional muzzle component removed.

FIG. 5 shows a top view of a dog wearing the dog anxiety relief bone conduction audio device, system with transducers positioned above the ears.

FIG. 6a shows the dog anxiety relief bone conduction audio device, system with transducer pedestals and transducers mounted to the dog collar using hook and loop material (e.g. Velcro).

FIG. 6b shows the dog anxiety relief bone conduction audio device, system with transducer pedestals and transducers mounted to the dog collar using hooks or snaps.

FIG. 6c shows the dog anxiety relief bone conduction audio device, system with transducer pedestals and transducers mounted to the dog collar using a flexible, adjustable slide positioning rail guide.

FIG. 7 shows a simple block diagram of the audio system components.

FIG. 8a shows the dog anxiety relief bone conduction audio device, system with a sound source unit, separate pre-amp unit, a separate amplifier unit, and transducer pedestals and transducers mounted to the dog collar component.

FIG. 8b shows the dog anxiety relief bone conduction audio device, system with a sound source unit with integrated pre-amp units and amplifier units, and transducer pedestals and transducers mounted to the dog collar component.

FIG. 8c shows the dog anxiety relief bone conduction audio device, system with a sound source unit, pre-amp units, and amplifier units integrated into the transducer pedestals and transducers mounted to the dog collar component.

FIG. 9a shows the dog anxiety relief bone conduction audio device, system dog collar component alternatively made from elastic materials eliminating the need for fasteners.

FIG. 9b shows the dog anxiety relief bone conduction audio device, system dog collar component alternatively utilizing a Velcro fastening closure.

FIG. 9c shows the dog anxiety relief bone conduction audio device, system dog collar component alternatively utilizing a hook or snap fastening closure.

FIG. 9d shows the dog anxiety relief bone conduction audio device, system dog collar component alternatively utilizing a snap in buckle closure.

FIG. 10a shows the dog anxiety relief bone conduction audio device, system dog collar component with muzzle component utilizing a snap in buckle closure.

FIG. 10b shows the dog anxiety relief bone conduction audio device, system dog collar component with muzzle component utilizing a hook or snap fastening closure.

### DETAILED DESCRIPTION OF THE INVENTION

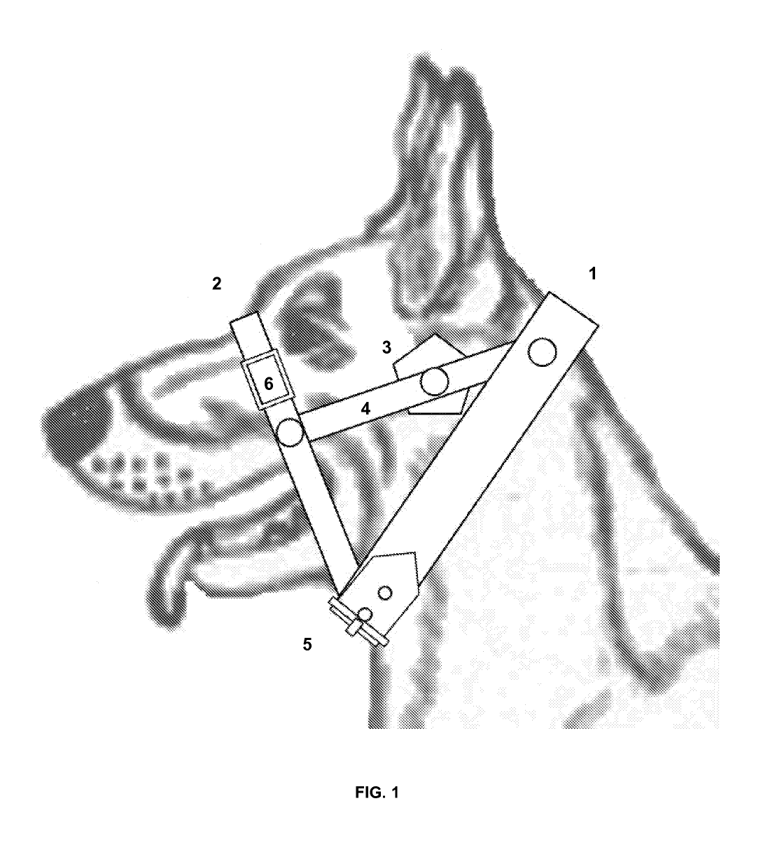

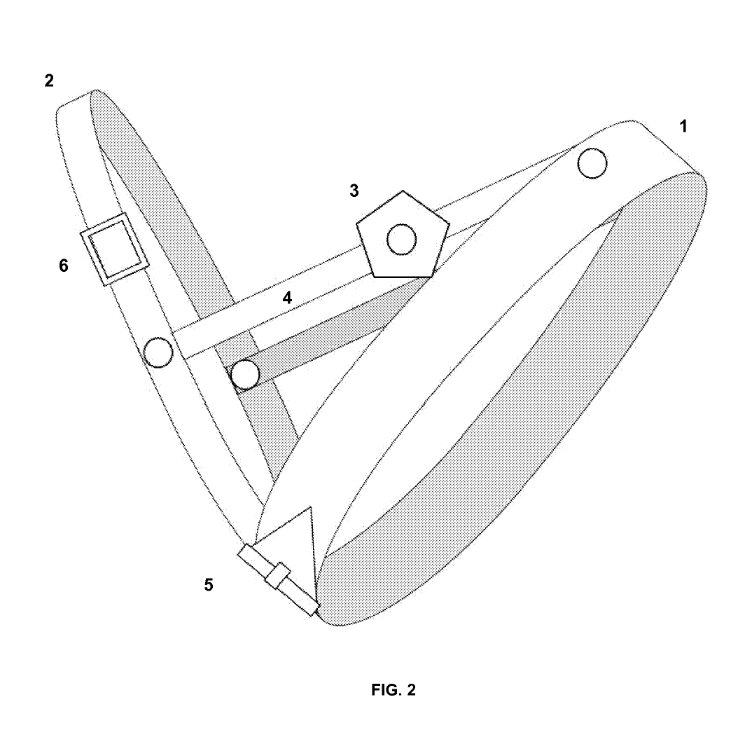

Referring now to the invention in more detail, in FIG. 1 and FIG. 2 there is shown the preferred embodiment of the dog anxiety relief bone conduction audio device, system as an adjustable dog collar component 1 and muzzle component 2 worn by a dog. In this embodiment, the transducer 3 is located on a cross piece 4 that connects the dog collar component 1 to the muzzle component 2 to allow optimum contact with the head of the dog as shown in FIG. 1. The dog collar component 1 can be adjusted for size using a buckle device 5 and will be about 10 to 28 inches in length and about 1 to 1.5 inches in width. The muzzle component 2 in FIG. 1 and FIG. 2 can be adjusted for size using the slide buckle 6 or removed by the dog's owner if desired. The muzzle component 2 will be about 10 to 20 inches in length and will be about 0.5 inches wide. The cross piece 4 will be made of impact resistant plastic or any other rigid natural or synthetic material suitable to hold the transducers 3 firmly in place and will vary in length to meet the dimensions of the dog collar component 1 and the muzzle component 2. Sealed, waterproof wiring (not shown) would be located inside the dog collar component for connecting to a signal source.

One of the major advantages of the dog anxiety relief bone conduction audio device, system is the capability to customize the configuration to the individual dog. Dogs vary greatly in their tolerance for objects attached on or around the head area. The dog's owner compensates for the individual dog's tolerances by adding or removing the muzzle component 2 and by moving the transducer 3 around the collar component until a configuration is found that is acceptable to the dog. The skull has many vibratory modes which allows placement of bone conductive transducers in multiple positions around the head without the loss of functionality.

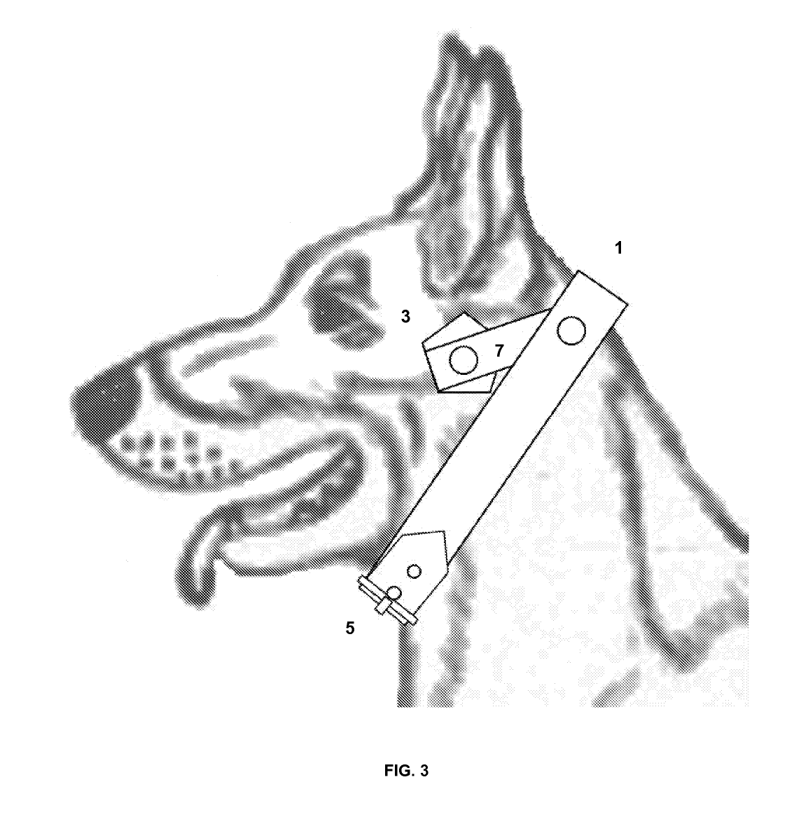

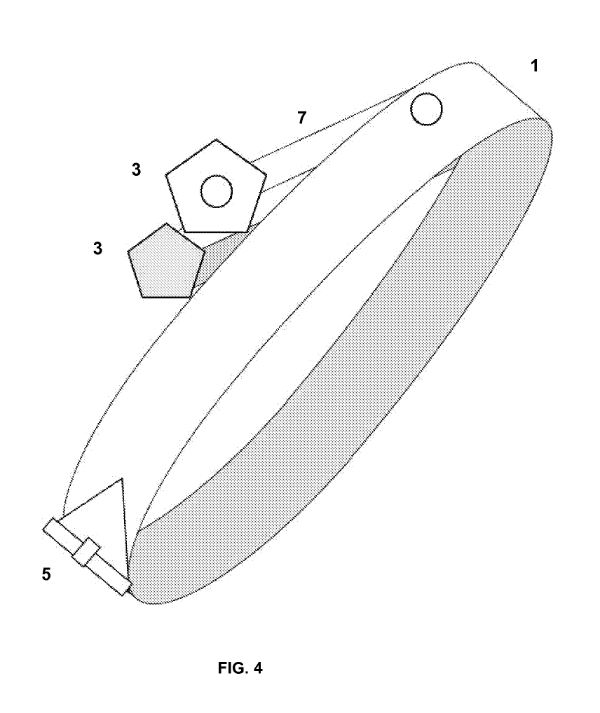

Another preferred configuration as shown in FIG. 3 and FIG. 4 is the dog anxiety relief bone conduction audio device, system with the muzzle component removed by the dog's owner. In this embodiment, the transducer 3 is mounted on a transducer pedestal 6 that attaches to the inside of the dog collar component 1 to allow optimum contact with the head of the dog as shown in FIG. 3. Sealed, waterproof wiring (not shown) would be located inside the dog collar component for connecting to a signal source.

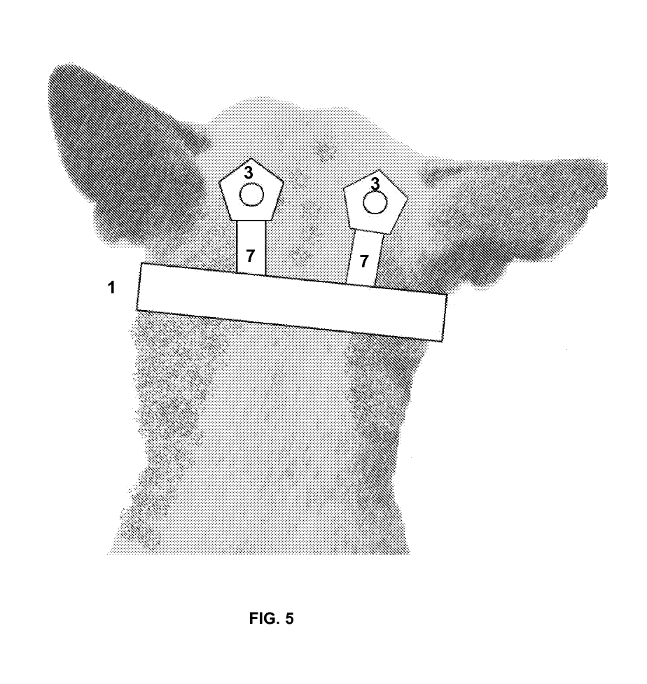

Another means for configuring the dog anxiety relief bone conduction audio device, system to maximize the comfort of the dog would be to reposition the transducers by repositioning the transducer pedestals around the dog collar component. FIG. 5 shows the transducers 3 mounted to the transducer pedestals 7 which have been attached to the dog collar component 1 at the top of the dog's head. The transducer pedestals 7 will be made of impact resistant plastic or any other rigid natural or synthetic material suitable to hold the transducers 3 firmly in place The unique vibratory pattern of a head is a product of the skull and brain complex geometry, mass and other acoustic properties and allows the transducers 3 to propagate sound from several locations around the skull.

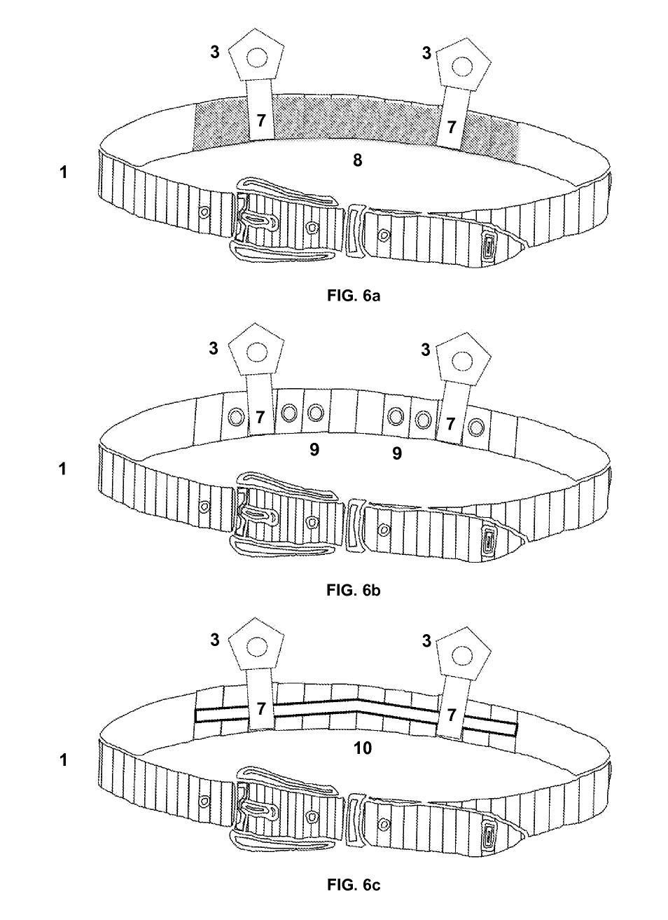

There are several embodiment of the dog anxiety relief bone conduction audio device, system dog collar component to allow for the repositioning of transducer pedestals. FIG. 6a show hook and loop material 8 inside the dog collar component 1 as the means to allow the dog's owner to remove and replace the transducer pedestals 7 with mounted transducers 3 in preferred positions around the dog collar component 1 for the dog's comfort. FIG. 6b shows the use of hooks or snaps 9 as the means to allow the dog's owner to reposition the transducer pedestals 7 with mounted transducers 3. Another means for repositioning would be to locate the transducer pedestals 7 with mounted transducers 3 by sliding them around the dog collar component 1 on a slide positioning rail guide 10 shown in FIG. 6c.

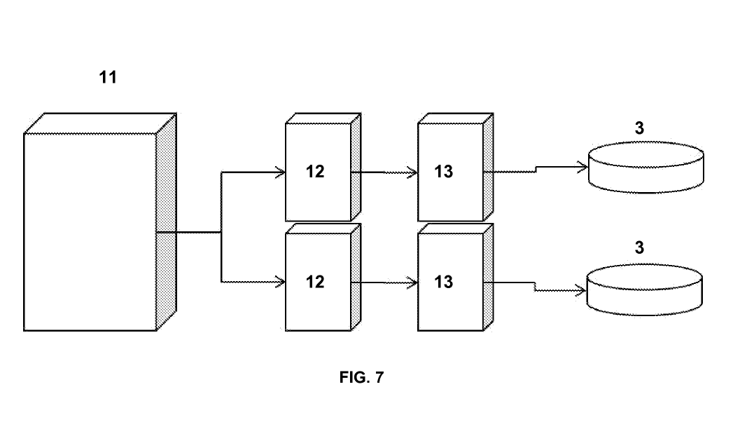

The therapeutic auditory stimulation music included in the dog anxiety relief bone conduction audio device, system can be Canine Lullabies or a similar music provider. The audio system will be comprised of commercially available components that are mounted to the dog collar component and may be connected together via wire or wirelessly. In order to maximize the sound quality of the musical signal, the sound source is amplified and split into two or three frequency channels. The sound source unit 11 shown in FIG. 7 is powered by a battery. A source signal is received from the sound source by the pre-amps 12. Amplifiers 13 further enhance the multi-channel signals. The dog's owner is able to adjust the volume until a comfortable level is achieved. At maximum volume, the device should be configured so that individuals near the dog should not be able to hear the sound.

The signal is sent to the appropriate transducer 3. These transducers 3 can be an Audiological Engineering Inc. device, a Radioear Corporation device, a device from Blatek Inc., or a similar device. The intended embodiment of the dog anxiety relief bone conduction audio device, system is to be able to operate in high humidity environments. As such, in most applications of the invention, the transducers should be waterproof. The transducer 3 preferably is waterproofed by rubberized or polymer coating.

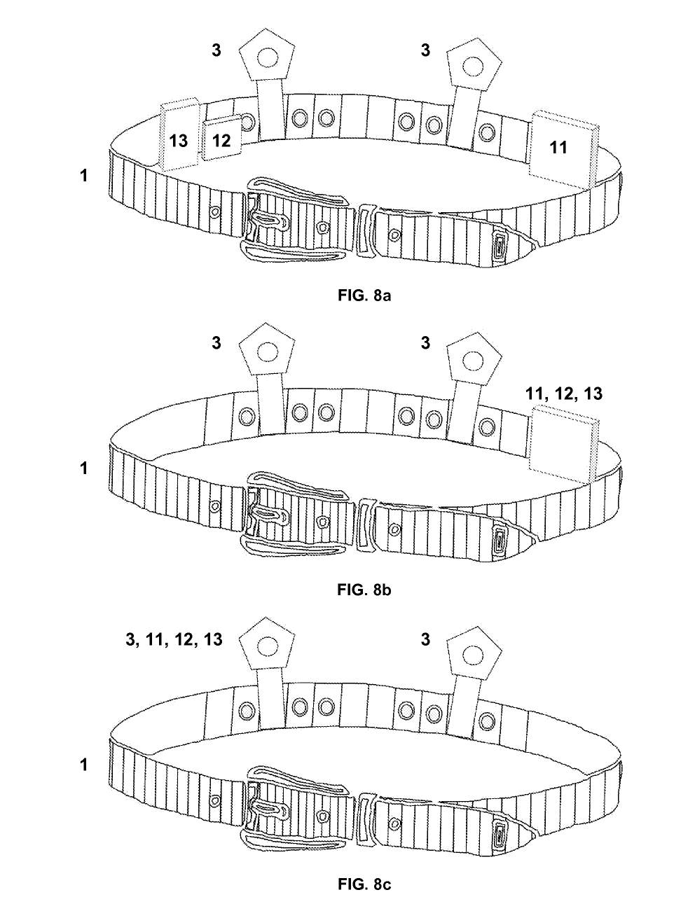

FIG. 8a further shows the dog anxiety relief bone conduction audio device, system with the sound source unit 11, the pre-amps 12, the amplifiers 13, and the transducers 3 as independent components mounted to the dog collar component 1. Another embodiment of the dog anxiety relief bone conduction audio device, system is to include the pre-amps 12 and the amplifiers 13 as part of the sound source unit 11 as shown in FIG. 8b. The sound source can be an MP3 player, CD player, or other portable musical device. FIG. 8c shows another embodiment of the dog anxiety relief bone conduction audio device, system that includes the sound source unit 11, the pre-amps 12 and the amplifiers 13 as part of the transducers, making it a fully integrated audio system.

Still in reference to FIG. 8a, and FIG. 8b, the sound source unit 11 is coupled to the dog collar component 1 by a wired connection or by a wireless connection. A wireless connection would comprise a sound source unit 11 that would communicate by transmitting and receiving the sound signals by radio, Bluetooth technology, or similar transmission means.

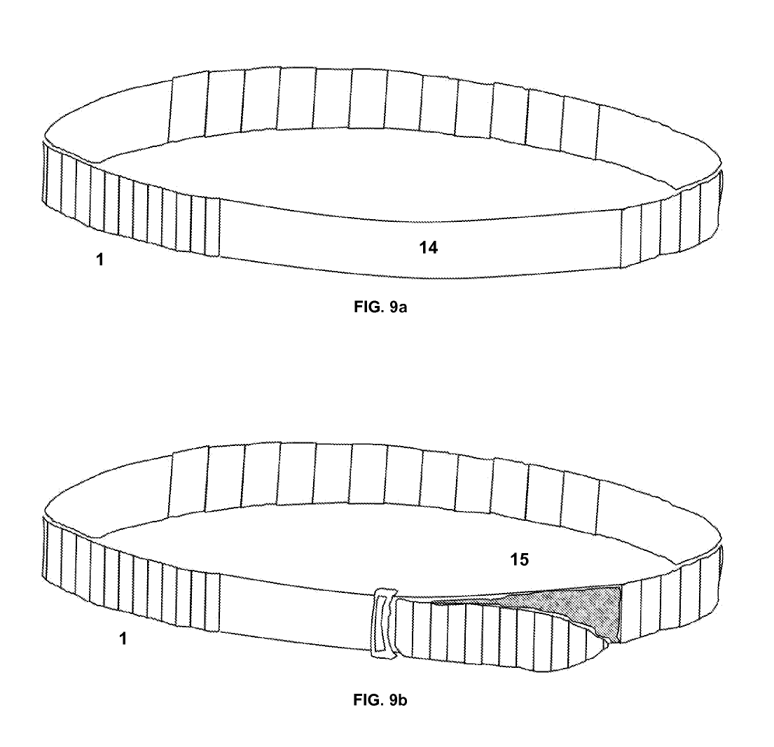

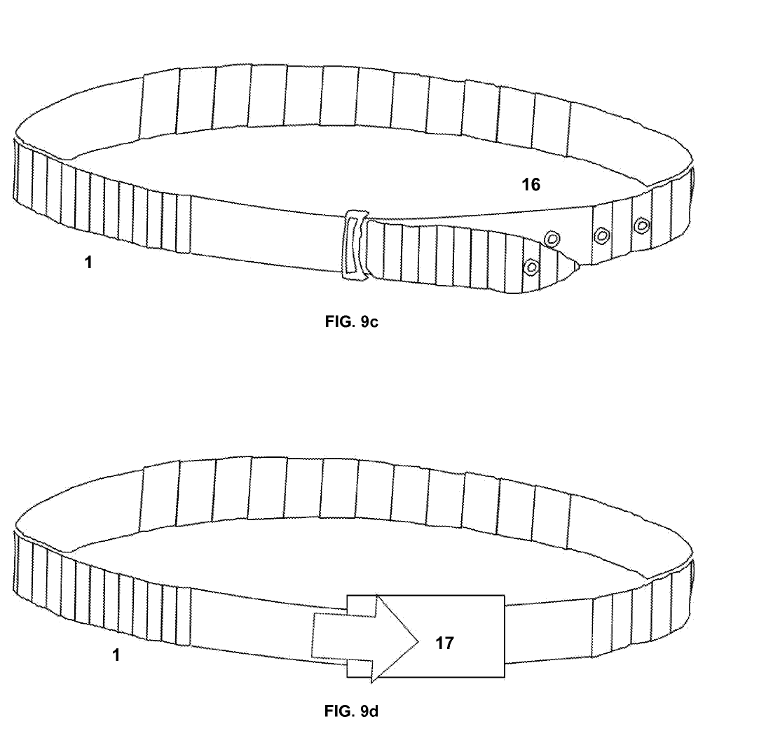

Adaptability and comfort of the dog are major features for the dog anxiety relief bone conduction audio device, system. The dog collar component and the adjustable muzzle component can be made using leather, cotton, or synthetic materials. A number of additional adjustable fastening devices can be used on the dog collar component to allow the dog's owner to ensure a proper fit for their dog. FIG. 9a shows a dog collar component 1 incorporating an elastic material 14 that would stretch over the dog's head and would require no closure device. A hook and loop (e.g. Velcro) fastener 15 is shown in FIG. 9b that would allow the dog's owner to adjust the dog collar component 1 to a fixed size. FIG. 9c shows the use of hooks or snaps 16 as the means to allow the dog's owner to adjust the dog collar component 1. A snap buckle device 17 is another alternative as a closure device for the dog collar component 1.

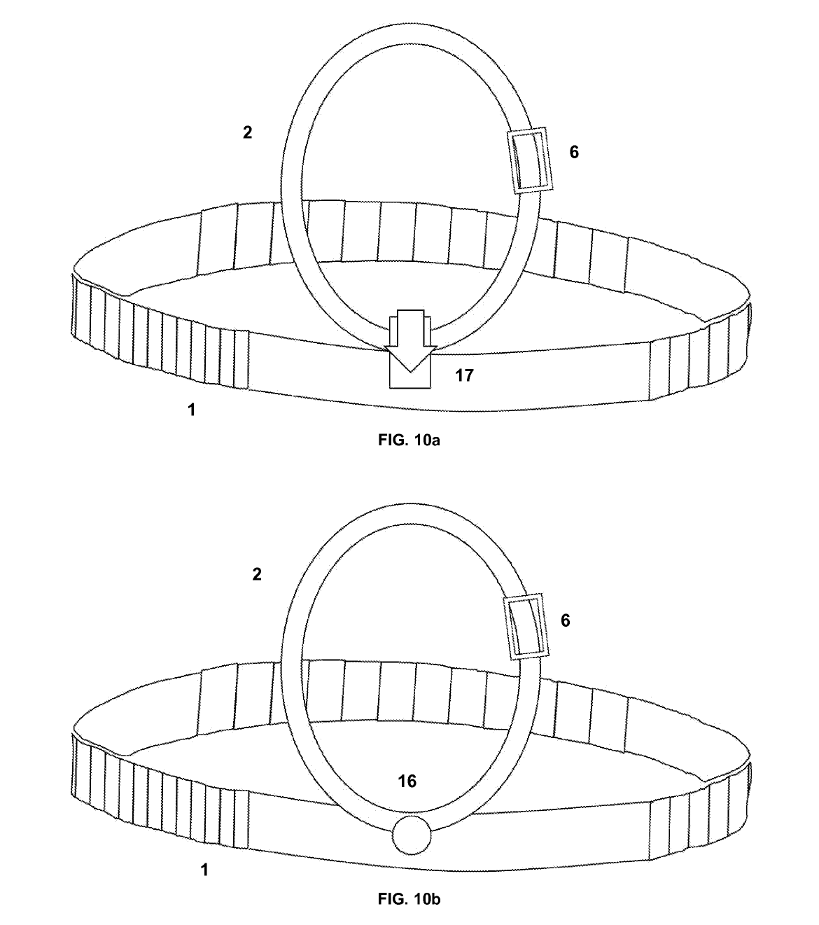

The ability to remove the muzzle component 2 makes the dog anxiety relief bone conduction audio device, system adaptable for dogs that do not have a tolerance for muzzle devices. The dog's owner can remove the muzzle component 2 from the dog collar component 1 by unfastening a snap buckle device 17 as shown in FIG. 10a. FIG. 10b shows another embodiment of the device that allows the dog's owner to remove the muzzle component 2 from the dog collar component 1 by unfastening hooks or snaps 16. Both FIG. 10a and FIG. 10b show a slide buckle 6 that allows a dog's owner to adjust the size of the muzzle component 2 to fit their dog.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

I claim:

1. A waterproof audio device and system that transmits sound via transcutaneous bone conduction to provide therapeutic auditory stimulation music to a dog, comprising: a) transducers for generating the music from musical signals; and b) a support for holding the transducers in vibratory contact with a dog's head, wherein each of the transducers is positionable at multiple locations on the support, wherein the support is an adjustable dog collar component and a removable adjustable muzzle component.

2. The audio device according to claim 1, further comprising a housing means for housing each of the transducers which includes a waterproofing polymeric material which covers each of the transducers.

3. The audio device according to claim 1, wherein at least one of the transducers is an ultrasonic transducer.

4. The audio device according to claim 1, wherein at least one of the transducers is a vibrotactile transducer.

5. The audio device according to claim 1, further including at least one amplifier coupled to one or more of the transducers for amplifying the musical signals.

6. The audio device according to claim 1, further comprising attachment features which attach the transducers to the collar structure.

7. The audio device according to claim 6, wherein that attachment features are attachment features selected from the group consisting of slide positioning guide features, hook features, snaps features and hook and loop fabric features.

8. The audio device of claim 1 further comprising a sound source for providing the musical signals to the transducers.

9. The audio device of claim 8 wherein the sound source provides the musical signals to the transducers through a wire connection.

10. The audio device of claim 8 wherein the sound source provides the musical signals to the transducers through a wireless connection.

11. The audio device of claim 8 wherein the sound source attaches to the dog collar.

12. The audio device of claim 8 wherein the sound source is selected from the group consisting of a disk player (e.g., CDs, DVDs, minidiscs, etc.), MP3 player, AM/FM radio, audio transceiver or other such devices known as personal audio devices.

13. The audio device of claim 1 wherein the collar is connected to a muzzle, and the transducers are positionable at multiple locations along a length of the band.

14. A therapeutic audio device, comprising: a) transducers that include a polymeric waterproofing cover and that produce an audio output; and b) a collar which fits around a dog's neck and holds the transducer in contact with a plurality of locations around the head of the dog, wherein the transducers are movable to different locations on the collar, and wherein the transducers generate an audio output transmitted to the dog through transcutaneous bone conduction.

15. The therapeutic audio device according to claim 14 wherein at the least one transducer can slide to different locations on the band.

16. The therapeutic audio device of claim 14 further comprising a sound source for providing audio signals that generate the audio output through transducers.

17. A method for a dog to hear music via transcutaneous bone conduction, comprising the steps of: a) supplying musical signals from a source to transducers each of which include a water proof housing at least partially formed from a polymeric material; b) contacting the transducers at positions on the dog's head using a collar that goes around the dog's head; and c) transmitting music through the dog's head by transcutaneous bone conduction through the polymeric material while the dog's head is wet.

18. The method recited in claim 17, further comprising a step of tuning the music.

19. The method of claim 18 wherein tuning the music comprises changing one or more of the positions of the transducers on the dog's head.

20. The method of claim 19 wherein changing the one or more of the positions of the transducers on the dog's head includes changing a position of one or more of the transducers on the collar.
